(19) United States
(12) Patent Application Publication
(10) Pub. No.: US 2020/0386425 A1
(43) Pub. Date: Dec. 10, 2020

(54) Mist Generating Device

(71) Applicant: CBC Co., Ltd., Tokyo (JP)

(72) Inventor: Yoshihisa Hayashi, Osaka (JP)

(21) Appl. No.: 16/464,234

(22) PCT Filed: Oct. 27, 2017
(86) PCT No.: PCT/JP2017/038963

§ 371 Date: May 24, 2019

(51) Int. Cl. F24F 6/16; F24F 6/14; A61M 11/00; A61H 35/00

(52) U.S. Cl. CPC F24F 6/16; F24F 6/14; A61M 2205/36; A61H 35/006; F24F 2006/146; A61M 11/00

(57) ABSTRACT

A mist generating device is provided with: a housing provided with a water tank; a shaft portion provided inside the housing; an impeller disposed so as to be rotatable in the horizontal direction with the shaft portion as the rotation axis; and a nozzle jetting the water in the water tank to the impeller. the water jetted from the nozzle collides with the impeller to thereby smash the jetted water to cause mist. The housing is provided with an outside air introducing port, which is provided with outside air introducing means for actively introducing outside air into the housing, the housing is provided with a mist releasing port, the mist releasing port is smaller in opening diameter than the outside air introducing port, and the inside of the housing can be pressurized by the active introduction of outside air into the housing by the outside air introducing means.

### TECHNICAL FIELD

The present invention relates to a mist generating device, and more particularly, relates to a mist generating device used for mist saunas and mist baths of elderly or sick persons' limbs.

### BACKGROUND ART

It is well known that exposure to mist which is atomized hot water or water promotes perspiration and encourages blood circulation even if the mist is low in temperature. It is also well known that exposure to such mist improves metabolism and improves the effect of water retention of the skin. In particular, from exposure, to mist, of the whole body or parts such as limbs of elderly and sick persons who are bedridden and cannot take a bath, the effect of relaxing the body and mind is expected, which is extremely useful. In taking a mist bath, if the particle diameter of the mist is large, mist particles in contact with the human body unite with each other to become a large-diameter water drop, which causes wetness to give unpleasantness and reduces the effect of the mist bath. For this reason, it has emerged that the particle diameter of the mist is desirably as small as possible.

Conventionally, various devices for realizing the mist bath have been proposed. For example, Patent Literatures 1 and 2 each disclose a mist generating device that is provided with an impeller having a plurality of blades arranged in a circle in the mode of traversing the rotation direction and causes jet flows jetted from a plurality of nozzles to collide with the blades to generate mist with a small particle diameter. In the mist generating device, water in a water tank provided inside the device is heated by a heater and the hot water in the water tank is jetted from the nozzles. This enables generation of mist suitable for mist bath, small in diameter and high in temperature.

### CITATION LIST

### Patent Literatures

[Patent Literature 1] Japanese Laid-Open Patent Publication No. 2016-67582

[Patent Literature 2] Japanese Patent Publication No. 6118563

### SUMMARY OF INVENTION

### Technical Problem

However, in the above-described mist generating devices disclosed in Patent Literatures 1 and 2, the mist generated by the collision between the jet flows from the nozzles and the blades is vaporized and the heat of vaporization is taken, so that the temperature of the mixed flow (mist flow) of the mist and the air released from the device is reduced. For this reason, in actuality, a mist flow of a temperature lower than an assumed desired temperature is released from the device. To solve this problem, it is considered to heat the water in the water tank to a higher temperature by a heater; however, doing this increases the amount of steam in the device to increase the amount of moisture in the mist flow released from the device, so that unpleasantness due to wetness is given to the person taking the mist bath. Moreover, since it is necessary to increase the amount of heating of the water in the water tank by the heater, cost also increases.

The present invention is made in view of the above-mentioned problem, and an object thereof is to enable generation of a mist flow suitable for mist baths, small in particle diameter, high in temperature and not giving unpleasantness such as wetness to the user without any increase in cost.

### Solution to Problem

To attain the above-mentioned object, the inventor has found, as a result of earnest researches, that by pressurizing the inside of the device, vaporization of the mist is prevented, the heat amount of the mist can be increased and as a consequence, the reduction in the temperature of the mist flow released from the device can be suppressed. Therefore, to attain the above-mentioned object, in the present invention, the mist generating device is provided with a structure for pressurizing the inside of the device.

Specifically, a mist generating device according to the present invention is provided with: a housing provided with a water tank storing water; a heater provided in the water tank for heating the water inside the water tank; a shaft portion provided inside the housing and extending in a vertical direction; an impeller disposed on the shaft portion so as to be rotatable in a horizontal direction with the shaft portion as a rotation axis; and a nozzle jetting the water in the water tank to the impeller, the impeller includes: a plurality of blades arranged in a rotation direction of the impeller and with which the water jetted from the nozzle collides; and an upper plate and a lower plate situated on upper ends and lower ends of the blades, respectively, and arranged so as to sandwich the blades, the water jetted from the nozzle collides with the blades to thereby smash the jetted water to cause mist and drive the impeller to rotate, the housing is provided with an outside air introducing port for introducing outside air into the housing and the outside air introducing port is provided with outside air introducing means for actively introducing outside air into the housing, the housing is provided with a mist releasing port for releasing the mist generated inside the housing to the outside of the housing, the mist releasing port is smaller in opening diameter than the outside air introducing port, and the inside of the housing can be pressurized by the active introduction of outside air into the housing by the outside air introducing means.

In the mist generating device according to the present invention, by causing the hot water in the water tank heated by the heater to collide with the impeller from the nozzles, mist high in temperature and small in particle diameter can be generated. Specifically, the mist with a comparatively small diameter scatters and floats inside the water tank, whereas the spray with a large diameter does not scatter upward and falls to the lower part of the water tank where water is stored. As a result, only the mist with a small diameter can be selectively taken out. Moreover, in the mist generating device according to the present invention, by a method such as actively introducing outside air into the device by the outside air introducing means and making the opening diameter of the mist releasing port smaller than that of the outside air introducing port, the pressure inside the housing of the device can be increased. For this reason, vaporization of the mist is prevented and the heat amount of the mist can be increased, so that the reduction in the temperature of the mist flow released from the device can be suppressed. As a result, mist suitable for mist baths, high in temperature and small in particle diameter can be obtained without a complicated structure or an increase in cost.

In the mist generating device according to the present invention, it is preferable that the opening diameter of the mist releasing port be not more than half the opening diameter of the outside air introducing port.

By doing this, the pressure inside the housing of the device can be further increased, so that the efficiency of suppressing the reduction in the temperature of the mist can be further improved.

In the mist generating device according to the present invention, it is preferable that the impeller be provided more than one in number so as to be arranged in the vertical direction on the shaft portion.

By doing this, the amount of mist generation can be increased without any increase in device size.

In the mist generating device according to the present invention, it is preferable that the shaft portion provided with the impeller be provided more than one in number and a plurality of nozzles be provided so as to jet water to the blades, respectively.

By doing this, the amount of mist generation can be increased, and further in this case, by providing a plurality of impellers on one shaft portion, the amount of mist generation can be significantly increased.

In the mist generating device according to the present invention, it is preferable that the mist releasing port be provided more than one in number on the housing.

By doing this, two or more persons can use the mist generating device at the same time; for example, two or more persons can take a mist bath at the same time.

### ADVANTAGEOUS EFFECTS OF INVENTION

With the mist generating device according to the present invention, mist suitable for mist baths, high in temperature and small in particle diameter can be obtained without a complicated structure or an increase in cost.

### BRIEF DESCRIPTION OF DRAWINGS

FIG. 1 is an exploded perspective view showing a mist generating device according to an embodiment of the present invention.

FIG. 2 is a longitudinal cross-sectional view showing the mist generating device according to the embodiment of the present invention.

FIG. 3 is a schematic view for explaining a mist generating mechanism in the mist generating device according to the embodiment of the present invention.

FIG. 4 is a schematic view showing a use pattern of the mist generating device according to the embodiment of the present invention.

FIG. 5 is a longitudinal cross-sectional view showing a mist generating device according to a modification of the embodiment of the present invention.

### DESCRIPTION OF EMBODIMENTS

Hereinafter, a mode for carrying out the present invention will be described based on the drawings. The following description of a preferred embodiment is essentially illustrative and does not intend to limit the present invention, application methods thereof or uses thereof.

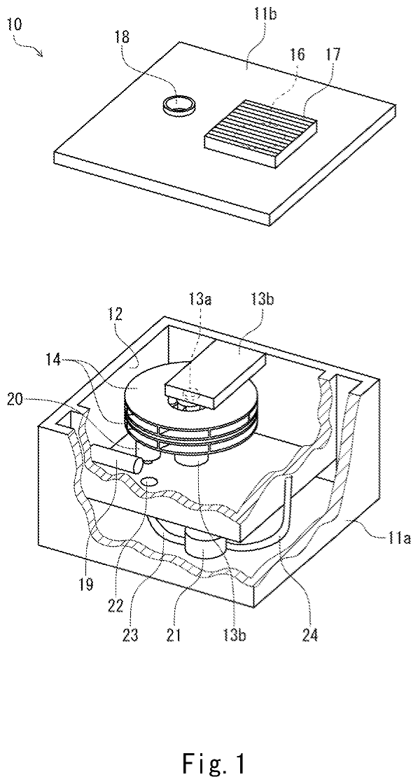

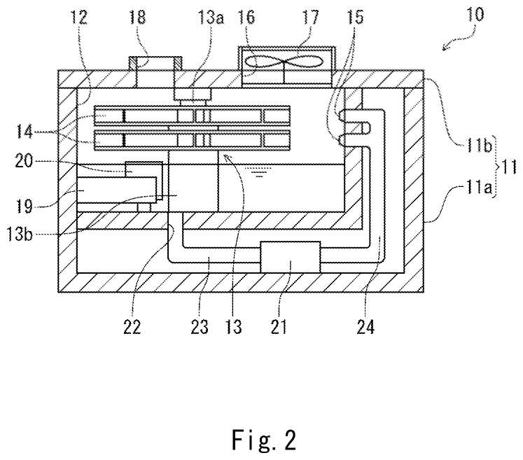

A mist generating device according to an embodiment of the present invention will be described with reference to FIGS. 1 to 3.

As shown in FIGS. 1 to 3, the mist generating device 10 according to the present embodiment includes a housing 11 formed of a body portion 11a and a lid portion 11b. The lid portion 11b is detachably attached to the body portion 11a with screws or the like. Inside the housing 11, a water tank 12 for storing water is provided. Inside the water tank 12, the following are provided: a shaft portion 13 extending in the vertical direction; impellers 14 disposed so as to be rotatable in the horizontal direction with the shaft portion 13 as the rotation axis; and nozzles 15 that jet the water in the water tank 12 to the impellers 14. Moreover, the lid portion 11b of the housing 11 is provided with an outside air introducing port 16 for introducing outside air into the housing 11, and the outside air introducing port 16 is provided with a fan 17 as the outside air introducing means for actively introducing outside air into the housing 11. Further, the lid portion 11b is provided with a mist releasing port 18 for releasing the mist generated inside the housing 11 to the outside of the housing 11.

The water tank 12 is provided with a heater 19 for heating the stored water and a float switch 20 for detecting that a predetermined amount of water is stored inside the water tank 12. The water inside the water tank 12 is heated by the heater 19, and the heated water is jetted from the nozzles 15 by a pump 21 provided inside the housing 11. Specifically, to the pump 21, the following are connected: a suction hose 23 connected to the water tank 12 through a suction hole 22 provided at the bottom of the water tank 12; and a sending-out hose 24 connected to the nozzles 15. Thus, the water inside the water tank 12 is sucked from the suction hose 23 through the suction hole 22 by the operation of the pump 21, passes through the sending-out hose 24, and is jetted from the nozzles 15.

The shaft portion 13 is formed of, specifically, a rotation shaft 13a extending in the vertical direction in the water tank 12 and bearing portions 13b provided at a bottom part and an upper part of the water tank 12, respectively, and supporting the lower end and the upper end of the rotation shaft 13a, respectively. On the rotation shaft 13a of the shaft portion 13, the impellers 14 rotatably attached in the horizontal direction are provided two in number so as to be arranged one above the other. The nozzles 15 are provided two in number in positions corresponding to the vertical positions of the two impellers 14, respectively. The impellers 14 are formed of a plurality of blades 14a arranged in the rotation direction of the impellers 14, and an upper plate 14b and a lower plate 14c situated on the upper ends and the lower ends of the blades, respectively, and arranged so as to sandwich the blades.

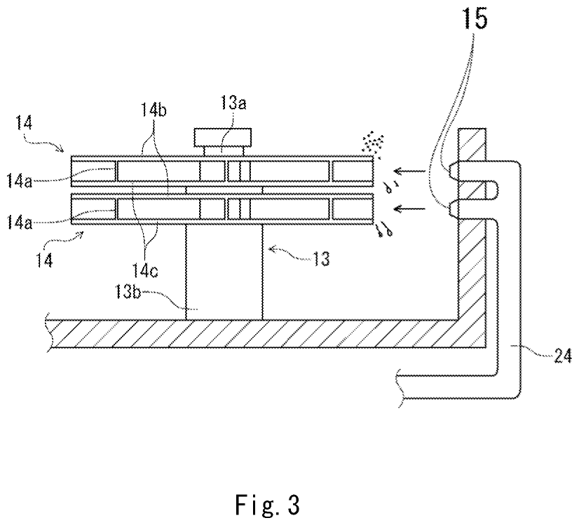

With the above-described structure of the mist generating device 10, as shown in FIG. 3, when water is jetted from the nozzles 15 to the impellers 14, the blades 14a of the impellers 14 receive the jetted water, so that the impellers 14 are driven to rotate in the horizontal direction. The water colliding with the blades 14a becomes mist and a spray including water droplets, and scatters in various directions. Thereby, mist is generated. Moreover, at this time, since the rotation shaft 13a of the impellers 14 is fixed in the vertical direction as described above and a centrifugal force therefore acts in the horizontal direction, the mist with a comparatively small diameter scatters and floats inside the water tank 12. On the other hand, the spray with a large diameter does not scatter upward and falls to the lower part of the water tank 12 where water is stored. As a result, only the mist with a small diameter can be selectively taken out.

In the mist generating device 10 according to the present embodiment, since the outside air introducing port 16 is provided with the fan 17 as the outside air introducing means as described above, outside air is introduced into the housing 11 by the operation of the fan 17. As a result, the mist generated and floating inside the housing 11 flows by the air flow introduced from the outside air introducing port 16 and released from the mist releasing port 18. Moreover, in the mist generating device 10 according to the present embodiment, the inside of the housing 11 is pressurized by the operation of the fan 17, and in particular, the opening diameter of the mist releasing port 18 is smaller than the opening diameter of the outside air introducing port 16.

In the mist generating device 10 according to the present embodiment, since the water tank 12 is provided with the heater 19 and water is heated by the heater 19 as described above, steam exists inside the water tank 12. In the conventional mist generating devices, as described above, mist is vaporized, so that the temperature of the released mist flow is reduced. However, in the mist generating device according to the present embodiment, since the inside of the housing 11 is pressurized as described above, vaporization of the mist is prevented and the heat amount of the mist can be increased, so that the reduction in the temperature of the mist flow released from the device can be suppressed.

In the present embodiment, although not shown, as the power source and wiring for operating the above-described fan 17, heater 19 and pump 21, the temperature setting device, the temperature sensor formed of a thermocouple, or the like, known ones are suitably adoptable.

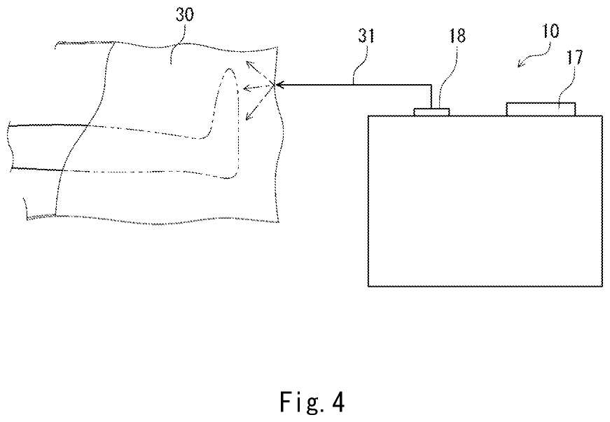

Next, an example of usage patterns of the mist generating device 10 according to the present embodiment will be described with reference to FIG. 4. Here, a use pattern where the mist generating device 10 is used for a mist bath of a foot will be described. As shown in FIG. 4, the mist generating device 10 can be used in such a manner that the mist releasing port 18 of the mist generating device 10 and a bag member 30 for the user to put his/her foot in are connected by a hose 31 so that the mist released from the mist releasing port 18 is introduced into the bag member 30. Doing this enables a mist bath of the user's foot with mist having a small particle diameter. While a use pattern of a use for a mist bath of a foot is shown in this example, a use for a mist bath of an arm or a mist bath of the whole body is enabled by changing the size of the bag member 30.

While the outside air introducing port 16 and the mist releasing port 18 are provided on the lid portion 11b of the housing 11 in the mist generating device 10 according to the present embodiment, they may be provided on the side of the body portion 11a. However, it is desirable that the mist releasing port 18 be provided in a part as upper as possible from the viewpoint of preventing a spray with a large diameter from reaching the mist releasing port 18. Moreover, the impellers 14 are provided two in number on the shaft portion 13, the number is not limited thereto; the number may be one, or three or more may be arranged. Here, the nozzles 15 of a number corresponding to the number of impellers 14 are provided. Moreover, in the mist generating device 10 according to the present embodiment, in order to appropriately pressurize the inside of the housing 11, it is desirable that the opening diameter of the mist releasing port 18 be not more than half the opening diameter of the outside air introducing port.

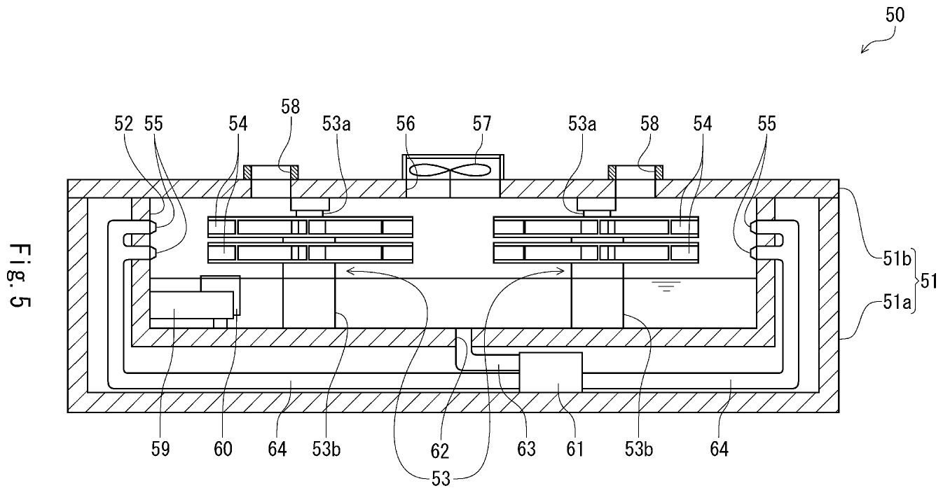

Next, a mist generating device 50 according to a modification of the present embodiment will be described. Comparing with the mist generating device 10 according to the above-described embodiment, the mist generating device 50 according to the present modification is different in that two shaft portions are provided, two impellers are provided on the shaft portions, respectively, and two mist releasing ports are provided. Hereinafter, the mist generating device 50 according to the present modification will be described with reference to FIG. 5. Here, descriptions of the same elements as those of the mist generating device 10 according to the above-described embodiment are omitted.

As shown in FIG. 5, in a water tank 52 inside a housing 51 of the mist generating device 50 according to the present modification, two shaft portions 53 are arranged in the horizontal direction. On each of the two shaft portions 53, two impellers 54 arranged one above the other are disposed so as to be rotatable in the horizontal direction. For this reason, a total of four nozzles 55 for jetting water to the impellers 54 to rotate them are provided one in each of the positions corresponding to the impellers 54. The four nozzles 55 are connected through a pump 61 and a sending-out hose 64 provided inside the housing 51, and water is jetted from the four nozzles 55 by the operation of the pump 61. In the present modification, since the number of impellers 54 is large as described above, the amount of mist generation can be increased.

Moreover, in the present modification, a lid portion 5 lb of the housing 51 is provided with two mist releasing ports 58. For this reason, two people can use one mist generating device 50 at the same time. Even in this case, in the present modification, since a number of impellers 54 for generating mist are provided as described above and the amount of mist generation is large, even if mist is released from the two mist releasing ports 58, the reduction in the amount of mist release from each can be prevented.

Moreover, in the present modification, like the mist generating device 10 according to the above-described embodiment, in the mist generating device 50 according to the present modification, the inside of the housing 51 is pressurized by the operation of a fan 57, and the opening diameter of the mist releasing ports 58 is smaller than the opening diameter of an outside air introducing port 56. In particular, in the present modification, by making the opening diameter of the outside air introducing port 56 larger, the amount of outside air introduction is increased, the inside of the housing 51 can be pressurized even though there are two mist releasing ports 58, and mist can be sufficiently released from the two mist releasing ports 58.

### EXAMPLES

An example for explaining the mist generating device according to the present invention in detail is shown below.

In the present example, the temperature of the fluid released from the device is compared between a mist generating device having the above-described structure of the present invention (example) and a mist generating device where no outside air introducing fan is provided and the inside of the housing of the device cannot be pressurized (comparative example).

Specifically, the mist generating device of the example is 1300 cm³ in the capacity of the housing, and has one shaft portion as shown in FIG. 1 to FIG. 3 and two impellers provided on the shaft portion. Moreover, the outside air introducing port has a fan as the outside air introducing means, outside air can be forcibly introduced into the housing of the device, and the mist releasing port is smaller in diameter than the outside air introducing port. On the other hand, the comparative example is different in that no fan as the outside air introducing means is provided although having a structure similar to that of the example.

With the devices of the example and the comparative example, the temperature of the fluid released from the mist releasing port was measured by the thermocouple provided at the exit of the mist releasing port under a condition where the temperature of the water inside the water tank was set at 55° C. to 80° C. and water is jetted to the impellers from the nozzles. The results are shown in Table 1.

[Table 1]

TABLE 1

| | | | | | | |
|---|---|---|---|---|---|---|
| Set temperature | 55° C. | 60° C. | 65° C. | 70° C. | 75° C. | 80° C. |
| Example | 41° C. | 44° C. | 47° C. | 50° C. | 52° C. | 56° C. |
| Comparative | 31° C. | 31° C. | 35° C. | 35° C. | 41° C. | 42° C. |
| example | | | | | | |

As shown in Table 1, it has become clear that in the mist generating device according to the present example, the temperature of the fluid (mist flow) released from the mist releasing port is higher than in the mist generating device of the comparative example. In the case where a mist bath of a foot was taken for twenty minutes at a set temperature of 70° C. by using these devices, when the mist generating device of the example was used, the perspiration effect was higher, less wetness was caused, and the feeling of use was excellent.

From the above, with the mist generating device according to the present invention, mist suitable for mist baths, high in temperature and small in particle diameter can be obtained without a complicated structure or an increase in cost, which is extremely useful.

### DESCRIPTION OF REFERENCE CHARACTERS

10, 50 Mist generating device

11, 51 Housing

12, 52 Water tank

13, 53 Shaft portion

14, 54 Impeller

15, 55 Nozzle

16, 56 Outside air introducing port

17, 57 Fan

18, 58 Mist releasing port

19, 59 Heater

20, 60 Float switch

21, 61 Pump

22, 62 Suction hole

23, 63 Suction hose

24, 64 Sending-out hose

1. A mist generating device comprising:
a housing provided with a water tank storing water;
a heater provided in the water tank for heating the water inside the water tank;
a shaft portion provided inside the housing and extending in a vertical direction;
an impeller disposed on the shaft portion so as to be rotatable in a horizontal direction with the shaft portion as a rotation axis; and
a nozzle jetting the water in the water tank to the impeller,
wherein the impeller includes:
a plurality of blades arranged in a rotation direction of the impeller and with which the water jetted from the nozzle collides; and
an upper plate and a lower plate situated on upper ends and lower ends of the blades, respectively, and arranged so as to sandwich the blades,
the water jetted from the nozzle collides with the blades to thereby smash the jetted water to cause mist and drive the impeller to rotate,
the housing is provided with an outside air introducing port for introducing outside air into the housing and the outside air introducing port is provided with outside air introducing means for actively introducing outside air into the housing,
the housing is provided with a mist releasing port for releasing the mist generated inside the housing to the outside of the housing, and
the mist releasing port is smaller in opening diameter than the outside air introducing port, and the inside of the housing can be pressurized by the active introduction of outside air into the housing by the outside air introducing means.

2. The mist generating device according to claim 1, wherein the opening diameter of the mist releasing port is not more than half the opening diameter of the outside air introducing port.

3. The mist generating device according to claim 1, wherein the impeller is provided more than one in number so as to be arranged in the vertical direction on the shaft portion.

4. The mist generating device according to claim 1, wherein the shaft portion provided with the impeller is provided more than one in number, and
a plurality of nozzles are provided so as to jet water to the blades, respectively.

5. The mist generating device according to claim 1, wherein the mist releasing port is provided more than one in number on the housing.
